(19) United States
(12) Patent Application Publication
(10) Pub. No.: US 2004/0151762 A1
(43) Pub. Date: Aug. 5, 2004

(54) Cough mitt

(76) Inventor: James H. Pridgen, Wilmington, NC (US)

Correspondence Address: WILLIAM J. MASON, MACCORD MASON PLLC, POST OFFICE BOX 1489, WRIGHTSVILLE BEACH, NC, 28480, US

(21) Appl. No.: 10/358,592

(22) Filed: Feb. 5, 2003

(51) Int. Cl. A61K 9/70; A61F 13/00

(52) U.S. Cl. 424/443

(57) ABSTRACT

A cough mitt for use in minimizing the aerosol spread of cold and flu viruses due to coughing and sneezing. The mitt includes an absorbent pad that is sized to be worn on the user's palm and attachment means to secure the pad in place. The pad may be formed of an inner absorbent layer, a replaceable outer layer, and a moisture impervious backing. A bactericide or virucide may be incorporated into at least one of the pads. The attachment means may be in the form of loops that fit around the wrist and a finger of the user.

### BACKGROUND OF THE INVENTION

(1) Field of the Invention

The present invention relates to a device for minimizing the spread of aerosolborne viruses, and in particular to a mitt with an aerosol absorbent pad that can be comfortably and conveniently supported in the palm of the user's hand, so that the user can cough or sneeze into the pad.

(2) Description of the Prior Art

A collection of respiratory symptoms caused by more than 200 rhinoviruses and generally referred to by the term “common cold” are normally experienced by the average adult 2-4 colds each year. Influenza or the “flu” is less common, but more serious. There is no cure for the common cold. Instead, a person acquiring a cold should preferably drink fluids and get plenty of bed rest to enable the body to fight off the infection.

Unfortunately, some individuals do not have the luxury of bed rest while experiencing a cold, generally due to the demands of their employment, or other activities that require them to interact with others during the duration of the cold. This interaction not only prolongs the cold, but promotes infection of others. One of the primary ways of transmitting cold or flu viruses is through coughing and sneezing, which produces an aerosol spray that can linger in the air for long periods of time at considerable distances from the source of the cough or sneeze. Other individuals then acquire the cold virus by inhaling the aerosol particles.

Educating people about the dangers of unprotected coughs and sneezes is the No. 1 influenza prevention method listed in the classic text on infectious diseases, “Control of Communicable Diseases in Man,” published by the American Public Health Association (APHA). Also needed is education of employers, so they realize that an employee on the job with a cold or flu may be a health risk to other workers. That seemingly loyal employee may cause absenteeism, lost productivity and lost profits as the disease spreads and more employees fall ill.

Generally, the primary education provided to employees, if the employees must come to work, is to cover the mouth and nose with disposable tissues when sneezing and coughing to minimize the spread of virus through aerosols, and also to frequently wash their hands with soap and warm water. However, it is not always possible for the person to reach a disposable tissue and place it over their nose and mouth when there is a sudden urge to cough or sneeze.

Therefore, there is a need for a device that can be used discretely by a person coughing or sneezing to trap virus contaminated aerosols. In particular, there is a need for a device that can be inconspicuously and conveniently carried by the user, while being immediately available to trap the aerosols in the event of a sudden urge to cough or sneeze.

### SUMMARY OF THE INVENTION

The present invention is directed to a device to address these needs. In general, the device of the present invention, referred to herein as a mitt, is comprised of a pad adapted to be worn on the palm of a user's hand, and an attachment means to hold the pad in place.

The pad in its basic construction is comprised of an absorbent pad layer that is capable of capturing aerosols from the cough or sneeze. The pad layer may be of various constructions. For example, at least a part of the pad layer may be of a nonwoven, fibrous material, or of a foam. The pad may be formed of a cellulosic substrate of the type used in the construction of disposable tissues. Preferably, the pad is from about 0.025 to about 0.25 inch, and preferably from about 0.05 to about 0.25 to about 0.125 inch, in thickness.

The pad may be formed of a single absorbent pad layer, or of an inner pad layer and an outer pad layer. When constructed of two layers, the user may replace the outer pad layer during use to refresh the pad without the necessity of replacing the entire pad. For example, the inner pad layer may have an outer face that is coated with a releasable adhesive that is adapted to releasibly adhere the outer pad layer in place during use. The pad layers may be of different thicknesses. For example, the ratio of the outer pad layer thickness to the inner pad layer thickness may be from about 1:4 to about 1:1. When reference is made herein to the “pad” it is to be understood that the term is used to encompass both a single pad or a pad made of multiple sections or layers.

The pad may also include a backing that is adapted to be positioned between the user's palm and the absorbent pad layer. Generally, the backing will be of the same dimensions as the pad, and will include an adhesive front surface to adhere the absorbent pad layer to the backing. If desired, the backing adhesive can be a releasable adhesive to permit releasable attachment of the pad to the backing, so that the pad can be replaced without replacement of the backing. Preferably, the backing is formed of a moisture impervious material, such as a plastic sheet, to prevent moisture from wicking from the pad to the user's palm. The backing thickness is not critical and may be, for example, about 1 mil to 4 mils.

If desired, the pad may also include an effective amount of one or more bactericides or virucides. Various bactericides and virucides suitable for this purpose will be apparent to one skilled in the art, and the exact amount used will vary depending upon the particular bactericide or virucide used.

The cough mitt also includes a means to releasibly attach the pad to the user's palm. The attachment means used should be such that the pad will be held securely in place during the user's normal activities, but which will permit easy replacement of the mitt when desired. For example, the attachment means may be comprised of one or more attachment loops that are secured around parts of the user's hand or wrist.

In one embodiment, the pad can be held in place by elastic loops. One loop can be attached to the back of the backing or pad and fit around the user's wrist. Another loop can extend from the front of the pad and fit around one of the user's fingers.

The mitt is used by placing the pad against the user's palm and securing it in place, e.g., by placing the loops around the user's wrist and finger. When the user has a sudden urge to cough or sneeze, he or she simply coughs or sneezes into the mitt, which is discretely positioned within the palm of the hand. When using a pad with upper and lower layers, the user can periodically peel off the upper layer and replace it with a fresh layer that is releasibly held in place by an adhesive on the upper surface of the lower layer. Thus, the use of a disposable tissue is not necessary, but the spread of cold virus through aerosols due to coughing or sneezing is avoided.

### BRIEF DESCRIPTION OF THE DRAWINGS

FIG. 1 is a perspective view of a user's hand with the cough mitt in position for use.

FIG. 2 is side view of a cough mitt with attachment loops.

FIG. 3 is a perspective view of a user's hand with an alternative cough mitt in position for use.

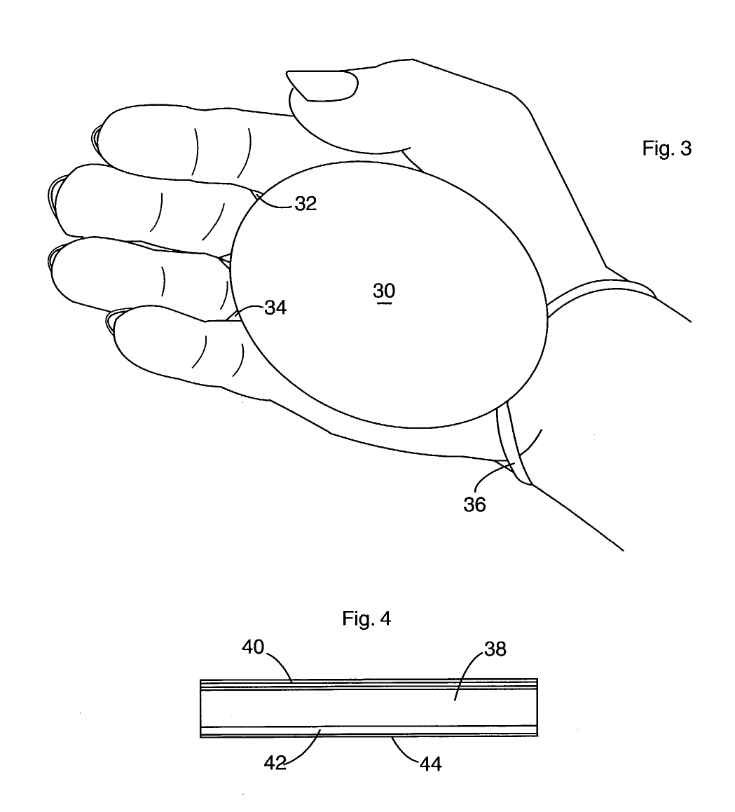

FIG. 2 is side view of a cough mitt with an adhesive attachment backing.

### DETAILED DESCRIPTION OF THE INVENTION

In the following description, terms such as horizontal, upright, vertical, above, below, beneath, and the like, are used solely for the purpose of clarity in illustrating the invention, and should not be taken as words of limitation. The drawings are for the purpose of illustrating the invention and are not intended to be to scale.

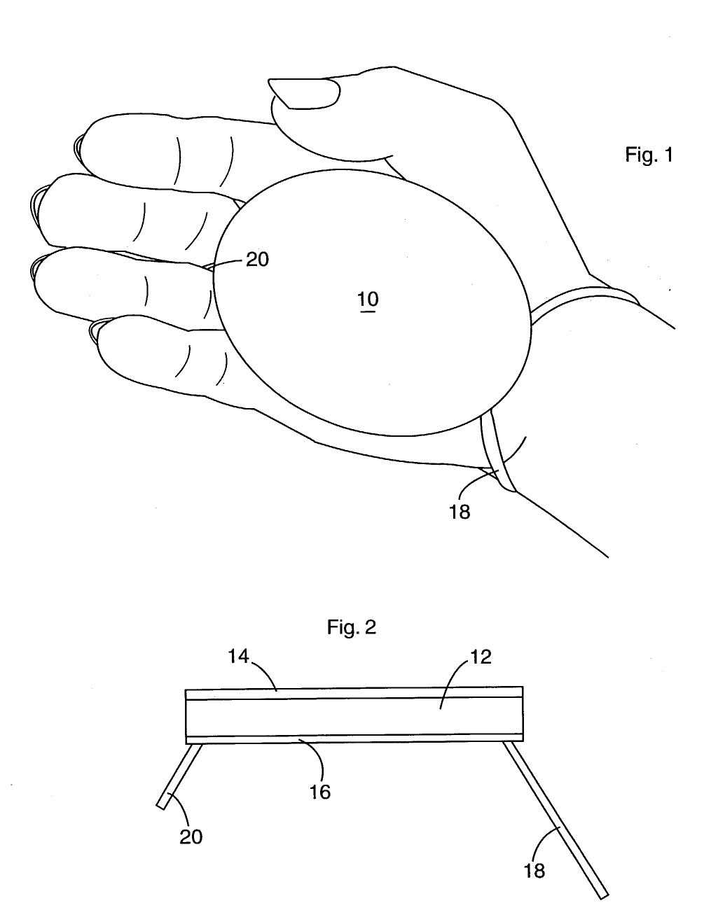

As shown in FIGS. 1 and 2 illustrating a preferred embodiment of the invention, cough mitt, generally 10, is comprised of an inner pad layer 12 with upper and lower surfaces, an upper pad layer 14 releasibly adhered to the upper surface of pad layer 12, and an impervious backing 16 releasibly adhered to the lower surface of pad layer 12. Pad layer 14, and preferably both pad layers 12 and 14, are absorbent. One or both absorbent pad layers may be formed of a nonwoven or foam material.

In the illustrations, pad layers 12 and 14 are shown as having the same area dimensions. However, it will be understood that pad layers 12 and 14 may be of different sizes. For example, pad layer 14 may have a larger surface area than pad layer 12, with only the central portion of pad layer 14 being adhered to the upper surface of pad layer 12. Either or both of pad layers 12 and 14 may contain a bactericide or virucide.

Pad layers 12 and 14 and backing 16, collectively referred to as the pad, are secured to the user's palm by an elastic wrist loop 18 extending from adjacent the rear of the pad, and an elastic finger loop 20 extending from adjacent the front of the pad. Loops 12 and 14 may be attached to one of layers 12 or 14, or to backing 16.

In use, backing 16 is positioned within the user's palm with loop 18 being secured around the user's wrist and loop 20 being secured around one of the user's fingers. The user can then cough or sneeze into the pad when necessary. Pad layer 14 can be periodically replaced with a fresh pad layer 14.

FIG. 3 illustrates an alternative cough mitt that includes two finger loops 32 and 34. It will be understood that multiple finger loops may be used, or that a finger loop can be sized to fit over more than one finger.

FIG. 4 illustrates an alternative cough mitt in which multiple absorbent peel-off layers 40 are attached to the upper surface of pad layer 38, so that each layer 40 can be peeled off to provide a clean surface. The lower surface on impermeable backing layer 42 is coated with an adhesive covered by peel-off covering 44. Instead of attachment loops, covering 44 can be removed and the adhesive used to releasibly secure the pad to the user's palm.

Certain modifications and improvements will occur to those skilled in the art upon a reading of the foregoing description. It should be understood that all such modifications and improvements have been deleted herein for the sake of conciseness and readability but are properly within the scope of the following claims.

What is claimed is:

1. A cough mitt adapted to be worn on the palm of a user's hand comprising:
a) an absorbent pad dimensioned to fit within a user's palm; and
b) attachment means for securing the pad on the palm of a user's hand.

2. The cough mitt of claim 1, wherein said pad is formed of a nonwoven absorbent material.

3. The cough mitt of claim 1, wherein said pad is comprised of an inner pad layer and an outer pad layer releasibly adhered to said inner pad layer.

4. The cough mitt of claim 1, wherein said pad is comprised of an absorbent layer and an impervious backing.

5. The cough mitt of claim 1, wherein said attachment means includes at least one loop adapted to fit around a part of a user's hand.

6. The cough mitt of claim 1, wherein said pad includes a bactericide or virucide.

7. The cough mitt of claim 1, wherein said pad is oval.

8. A cough mitt adapted to be worn on the palm of a user's hand comprising:
a) an absorbent pad dimensioned to fit within a user's palm, said pad including an inner pad layer, an outer pad layer, and an impervious backing; and
b) attachment means for securing the pad on the palm of a user's hand.

9. The cough mitt of claim 8, wherein said inner layer includes an upper surface, and said outer pad layer is releasibly adhered to said inner pad layer upper surface.

10. The cough mitt of claim 8, wherein at least one of said pad layers is formed of nonwoven absorbent material.

11. The cough mitt of claim 8, wherein at least one of said pad layers is formed of foam absorbent material.

12. The cough mitt of claim 8, wherein said attachment means includes at least one loop adapted to fit around a part of a user's hand.

13. The cough mitt of claim 8, wherein said pad is oval.

14. A cough mitt adapted to be worn on the palm of a user's hand comprising:
a) an absorbent pad dimensioned to fit within a user's palm; and
b) loops adapted to fit around parts of the user's hand to secure the pad on the palm of a user's hand.

15. The cough mitt of claim 14, including an inner pad layer with upper and lower surfaces, an outer pad layer releasibly adhered to said inner pad layer upper surface, and an impervious backing releasibly adhered to said inner pad layer lower surface.

16. The cough mitt of claim 14, wherein said pad is formed of nonwoven absorbent material.

17. The cough mitt of claim 14, wherein at least one of said pad layers is formed of foam absorbent material.

18. The cough mitt of claim 14, wherein said pad includes a bactericide or virucide.

19. The cough mitt of claim 14, wherein said pad is oval.

20. The cough mitt of claim 14, wherein said pad includes an inner absorbent pad layer and an outer absorbent pad layer adhered to said inner absorbent pad layer, the ratio of the thickness of said outer pad layer to the thickness of said inner pad layer being from about 1:4 to about 1:1, the combined thickness of said inner and outer pad layers being from about 0.025 inch to about 0.25 inch.
